(19) United States
(12) Patent Application Publication
(10) Pub. No.: US 2019/0365540 A1
(43) Pub. Date: Dec. 5, 2019

(54) KINK RESISTANT MULTIPLE LUMEN PRESSURIZED FLUID TUBE

(71) Applicant: FIDELIS MEDICAL LTD., Tel Aviv (IL)

(72) Inventor: Chen Sulami, Hadera (IL)

(73) Assignee: FIDELIS MEDICAL LTD., Tel Aviv (IL)

(21) Appl. No.: 15/993,700

(22) Filed: May 31, 2018

(51) Int. Cl. A61F 2/26

(52) U.S. Cl. CPC A61F 2/26; A61F 2230/0091; A61F 2230/0069; A61F 2250/0003; A61F 2250/0067; A61F 2230/0034; A61F 2230/0008

(57) ABSTRACT

An inflatable prosthesis includes a tubular body made of an elastomeric material, and at least two longitudinal lumens formed in the elastomeric material of the tubular body, which extend through an entire length of the tubular body along a longitudinal axis of the tubular body. The lumens are separated from each other by a wall. A coil is embedded in the elastomeric material of the tubular body. The coil surrounds the lumens.

### FIELD OF THE INVENTION

The present invention relates generally to tubes in which pressurized fluid flows, and particularly to a multiple lumen tube for pressurized fluid flow, which may form part of a prosthesis, such as but not limited to, an inflatable penile prosthesis.

### BACKGROUND OF THE INVENTION

Inflatable penile prostheses are used in the treatment of erectile dysfunction (ED) or impotence. An example of an inflatable penile prosthesis is described in PCT Patent Application PCT/IB2016/057892. The penile prosthesis is implantable within the penis and achieves a penile erection upon fluid flowing from a fluid reservoir and inflating an inflatable tubular member. An axial penile pump of such a prosthesis requires both inlet for low pressure and outlet for high pressure in order to evacuate fluid from the reservoir to implant cylinders and achieve erection. The high pressure lumen also enables the deflation of the penile cylinders back to the reservoir for returning to the flaccid state.

### SUMMARY OF THE INVENTION

The present invention, relates, in some embodiments thereof, to a multiple lumen tube for pressurized fluid flow, which may form part of a prosthesis, such as but not limited to, an inflatable penile prosthesis. The tubes are made of an elastomeric material with at least two longitudinal lumens formed in the elastomeric material. The lumens are surrounded by a coil embedded in the elastomeric material. The coil helps prevent the tube from kinking.

According to an aspect of the present invention, there is provided apparatus including a tubular body made of an elastomeric material, at least two longitudinal lumens formed in the elastomeric material of the tubular body, which extend through an entire length of the tubular body along a longitudinal axis of the tubular body, the lumens being separated from each other by a wall, and a coil embedded in the elastomeric material of the tubular body, the coil surrounding the lumens.

Without limitation, each of the lumens may be oval shaped.

Without limitation, each of the lumens may be oval shaped with a major length tilted at an angle with respect to the longitudinal axis of the tubular body. For example, each of the lumens may be oval shaped with a major length in a range of 1.3-1.9 mm and a minor length in a range of 0.4-0.9 mm. A thickness of the wall may be in a range of 0.2-0.5 mm at a center of the tubular body.

The tubular body may be coated with a hydrophilic material. The tubular body may be coated with an antibiotic material.

A pressurizing fluid may be fluidly connected to at least one of the lumens.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a perspective illustration of a multiple lumen tube, constructed and operative in accordance with a non-limiting embodiment of the invention;

FIG. 2 is a front-view illustration of the multiple lumen prosthesis;

FIG. 3 is a side-view illustration of the multiple lumen prosthesis;

FIG. 4 is a simplified illustration of the prosthesis in use as a penile prosthesis; and

FIGS. 5A-5D are simplified illustrations of multiple lumen tubes, constructed and operative in accordance with other non-limiting embodiments of the invention, with different cross-sectional shapes of lumens and different configurations of coils.

### DETAILED DESCRIPTION

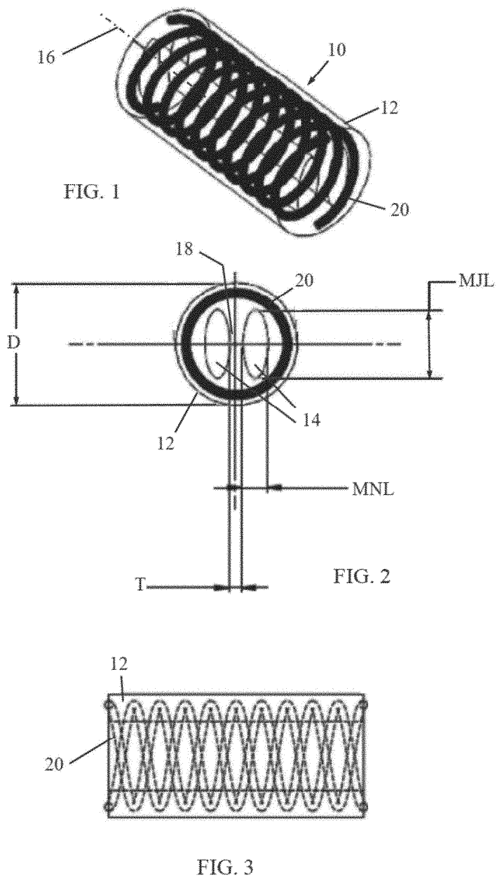

Reference is now made to FIG. 1, which illustrates a multiple lumen tube 10, constructed and operative in accordance with a non-limiting embodiment of the invention. The tube 10 includes a tubular body 12 made of an elastomeric material, such as but not limited to, polyurethane or silicone with a hardness of 50 Shore A. Without limitation, tubular body 12 may have a diameter D in a range of 2.7-3.6 mm.

At least two longitudinal lumens 14 are formed in the elastomeric material of tubular body 12. The lumens extend through the entire length of tubular body 12 along a longitudinal axis 16 of body 12. In the illustrated embodiment, each lumen 14 has a cross-section which is oval shaped with a major length MJL, without limitation, in a range of 1.3-1.9 mm and a minor length MNL, without limitation, in a range of 0.4-0.9 mm. In one embodiment, the major length is perpendicular to the longitudinal axis 16 of body 12. Alternatively, the major length may be tilted at an angle other than 90° to the longitudinal axis 16 of body 12.

The lumens 14 are separated from each other by a wall 18. Without limitation, the thickness T of wall 18 is in a range of 0.2-0.5 mm at the center of tubular body 12, which is the point of minimum thickness of wall 18.

The lumens 14 are surrounded by a coil 20 embedded in the elastomeric material of tubular body 12. Coil 20 may be made from an elastic, flexible material, such as but not limited to, nylon, nitinol and others.

Tubular body 12 may be coated with a hydrophilic material to facilitate movement of the tube after implantation, and/or with an antibiotic material to prevent possible infections following implantation.

Reference is now made to FIG. 4, which illustrates tube 10 in fluid connection with a penile prosthesis 30, such as but not limited to, an inflatable penile prosthesis described in PCT Patent Application PCT/IB2016/057892. Penile prosthesis 30 includes inflatable cylinders 32 implanted within the corpus cavernosa. Tube 10 is fluidly connected to a pressurizing fluid 22 (such as but not limited to, water or saline solution) that flows from a fluid reservoir or pump 24. Tube 10 directs the flow of fluid to the cylinders 32 of penile prosthesis 30. Both lumens of tube 10 may be used for inward flow of the fluid 22 to achieve an erection and outward flow to return to a flaccid state; alternatively, just one of the lumens may be inflated for erection. In one embodiment, one of the lumens of tube 10 may be used for flow of low pressure fluid and the other lumen may be for high pressure fluid flow from the reservoir/pump 24 to the cylinders 32 to achieve erection. The high pressure lumen also enables the deflation of the cylinders 32 back to the reservoir/pump 24 for returning to the flaccid state.

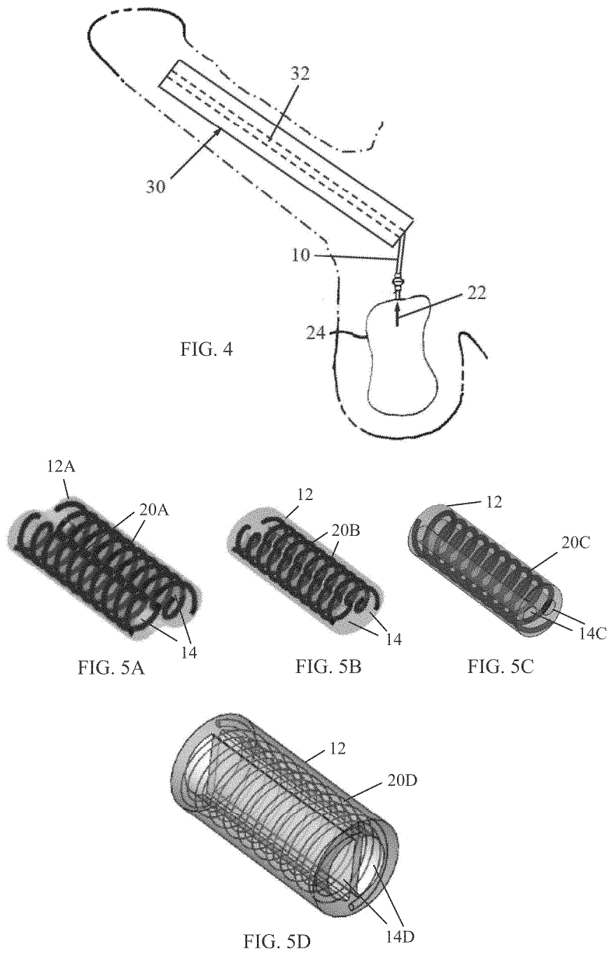

Reference is now made to FIGS. 5A-5D, which illustrate other multiple lumen tubes, in accordance with other non-limiting embodiments of the invention, with different cross-sectional shapes of lumens and different configurations of coils.

In FIG. 5A, each lumen 14 is surrounded by its own dedicated coil 20A and the tubular body 12A is not cylindrical but rather conforms to the outer contours of each coil 20.

In FIG. 5B, each lumen 14 is surrounded by its own dedicated coil 20B and the tubular body 12 remains cylindrical; there is more material surrounding the coils in FIG. 5B than in FIG. 5A.

In FIG. 5C, each lumen 14C has a circular cross-sectional shape and both are surrounded by coil 20C (alternatively each lumen could have its own dedicated coil).

In FIG. 5D, each lumen 14D has a D-shaped cross-sectional shape and both are surrounded by coil 20D (alternatively each lumen could have its own dedicated coil).

What is claimed is:

1. Apparatus comprising:
a tubular body made of an elastomeric material;
at least two longitudinal lumens formed in said elastomeric material of said tubular body, which extend through an entire length of said tubular body along a longitudinal axis of said tubular body, said lumens being separated from each other by a wall; and
a coil embedded in said elastomeric material of said tubular body, said coil surrounding at least one of said lumens.

2. The apparatus according to claim 1, wherein each of said lumens has an oval shaped cross-section.

3. The apparatus according to claim 1, wherein each of said lumens has an oval shaped cross-section with a major length tilted at an angle with respect to said longitudinal axis of said tubular body.

4. The apparatus according to claim 1, wherein each of said lumens has an oval shaped cross-section with a major length in a range of 1.3-1.9 mm and a minor length in a range of 0.4-0.9 mm.

5. The apparatus according to claim 1, wherein a thickness of said wall is in a range of 0.2-0.5 mm at a center of said tubular body.

6. The apparatus according to claim 1, wherein said tubular body is coated with a hydrophilic material.

7. The apparatus according to claim 1, wherein said tubular body is coated with an antibiotic material.

8. The apparatus according to claim 1, wherein a pressurizing fluid is fluidly connected to at least one of said lumens.

9. The apparatus according to claim 8, wherein said lumens of said tubular body are fluidly connected to cylinders of a penile prosthesis.

10. The apparatus according to claim 1, wherein each of said lumens has a D-shaped cross-section.

11. The apparatus according to claim 1, wherein each of said lumens has a circular cross-section.

12. The apparatus according to claim 1, wherein said coil surrounding both said lumens.

13. The apparatus according to claim 1, wherein a first coil surrounds one of said lumens and a second coil surrounds another of said lumens.
